(19) United States
(12) Patent Application Publication
(10) Pub. No.: US 2012/0271081 A1
(43) Pub. Date: Oct. 25, 2012

(54) METHOD FOR THE PRODUCTION OF C10+ HYDROCARBONS FROM HETEROATOMIC ORGANIC COMPOUNDS

(75) Inventors: Nikolai Nesterenko, Nivelles (BE); Delphine Minoux, Nivelles (BE); Sander Van Donk, Sainte-Adresse (FR); Jean-Pierre Dath, Beloeil Hainaut (BE)

(73) Assignee: TOTAL RAFFINAGE MARKETING, Puteaux (FR)

(21) Appl. No.: 13/501,408

(22) PCT Filed: Oct. 13, 2010
(86) PCT No.: PCT/FR10/52169

§ 371 Date: Jun. 29, 2012

(30) Foreign Application Priority Data
Oct. 13, 2009 (FR) 09 57158

(51) Int. Cl. C07C 2/88; C07C 2/04

(52) U.S. Cl. 585/315; 585/328

(57) ABSTRACT

The invention relates to a process for producing distillate from a charge of heteroatomic organic compounds comprising at least one heteroatom chosen from oxygen, sulfur and halogen, alone or in combination, in which the treatment of the charge comprises at least one step of conversion of the heteroatomic organic compounds into olefins performed in a first conversion zone, and, in at least a second oligomerization zone, a step of oligomerization of olefins originating at least partly from the conversion zone, in the presence of at least 0.5% by weight of oxygenated compounds, in order to produce a distillate. By virtue of the presence of oxygenated compounds during the oligomerization, this process makes it possible to improve the yield of distillate, making it possible to obtain a higher degree of oligomerization relative to the oligomerization of the same charge under the same reaction conditions.

The invention relates to a process for producing distillates from heteroatomic organic compounds, in particular from oxygenated compounds originating especially from biomass.

The term “distillate” means hydrocarbons containing 10 or more carbon atoms, middle distillates comprising from 10 to 20 carbon atoms and distilling in the temperature range from 145° C. to 350° C. Among the distillates, C10-C12 olefins (jet fuel) and C12+ olefins (diesel) will especially be distinguished.

Heteroatomic organic compounds (referred to hereinbelow as compounds “X”) are intermediate compounds that enable the upgrading of biomass, natural gas, charcoal, etc. This upgrading may be performed, for example, by synthesis of methanol or chloromethane.

Typically, the processes for converting these heteroaromatic organic compounds into olefins (“X To Olefin” (XTO) processes) enable the production of highly olefinic products, containing up to 92% by weight (based on carbon) of C2-C8 olefins containing a majority of light C2-C4 olefins. In particular, the content of C2-C3 olefins may be up to 50 to 55% by weight.

Optimization of the C4+ yield starting with oxygenated compounds in one run (one-through) leads to a substantial loss of carbon in the form of paraffins and aromatics, which are suitable only for the gasoline pool, thus decreasing the content of olefins in the effluent of the XTO process.

C2-C3 olefins, in particular ethylene, undergo little reaction under typical oligomerization conditions. Whereas butanes, pentenes and heavy olefins (comprising six or more carbon atoms) are converted to a degree of from 50% to 95% by weight, only 10% to 20% by weight of ethylene is converted on an oligomerization catalyst of acidic type. The catalytic oligomerization of ethylene takes place at higher temperatures and lower pressures. Under such conditions, the degree of conversion of ethylene is considerably increased, and the oligomerization of the light (C2-C4) olefins is virtually total, making it possible to produce an olefinic gasoline comprising hexene, heptene, octene and other hydrocarbons in good yields. However, under such conditions, a large part of the C4 is converted into aromatic hydrocarbons.

Thus, an oligomerization treatment of this type of charge leads to a low yield of distillate and necessitates a highly degree of recycling.

Processes exist combining a step of conversion of methanol into olefins and an oligomerization step.

Some of these processes especially use selective catalysts of zeolite type, such as the Mobil Olefins to Gasoline and Distillate (MOGD) process. The products obtained from butenes are trimers and tetramers, characterized by a low degree of branching.

Another similar process described in document US 2009/0 050 531 (=WO 2006/076 942) makes it possible to produce gasoline and diesel in a ratio of approximately 1:4. This process comprises a first step in which a gaseous mixture comprising methanol and/or DME and/or other oxygenated compounds and water vapor are converted into olefins, oligomerized at high-pressure in a second step in order to form heavier C5+ and preferably C10-C20 olefins. The production of olefins in the first step is performed in the presence of a gaseous stream composed essentially of saturated hydrocarbons which are separated from the effluents of the second step and recycled into the first step. The production of olefins is performed in the second step in the presence of a stream of water vapor, which is separated from the effluents of the first step and sent into the second step. The process described is thus a process for obtaining heavier olefins, but is not a process for producing distillates.

There is thus a need to improve the processes for transforming heteroatomic organic compounds (X) into distillate-rich fuel, substantially free of heteroatoms.

The Applicant has discovered a novel two-step process for transforming heteroatomic organic compounds, especially oxygenated compounds, into distillate, comprising a first step of converting the heteroatomic organic compounds into olefins and a second step of oligomerization of the olefins, containing at least part of the olefins thus formed, in the presence of oxygenated compounds.

In particular, the Applicant has discovered that the presence of oxygenated compounds during the oligomerization makes it possible to improve the yield of distillate, making it possible to obtain a higher degree of oligomerization relative to the oligomerization of the same charge under the same reaction conditions.

The process according to the invention, which is a process in several steps and a continuous process, makes it possible to improve the conversion of heteroatomic compounds into distillate.

A first subject of the invention is thus a process for producing distillate from a charge of heteroatomic organic compounds, in which the treatment of the charge comprises at least one step of conversion of the heteroatomic organic compounds into olefins performed in a first conversion zone, and, in at least a second oligomerization zone, a step of oligomerization of olefins originating at least partly from the conversion zone, in the presence of at least 0.5% by weight of oxygenated compounds, in order to produce a distillate.

By combining an olefin conversion zone and an oligomerization zone, the process according to the invention thus makes it possible to obtain an oligomerization-optimized charge, rich in C2-C8, improving the yield of distillate and also the carbon yield (carbon efficiency), which is important for GTL (Gas to Liquid), CTL (Coal to Liquid) and BTL (Biomass to Liquid) technologies.

Moreover, the presence of water precursors (the oxygenated compounds) during the oligomerization of olefins makes it possible to reduce the amounts of cracked products in the oligomerization zone. It also makes it possible to reduce the degree of deactivation of the catalysts used in the oligomerization zone and to limit the competing reactions with heavy olefins. In the present invention, water is formed in situ by dehydration of the oxygenated compounds.

Charge

The charge in the process according to the invention comprises heteroatomic organic compounds, which are organic compounds comprising at least one heteroatom chosen from oxygen, sulfur and halogen, alone or in combination.

The oxygenated organic compounds contain at least one oxygen atom, such as aliphatic alcohols, ethers, carbonyl compounds (aldehydes, ketones, carboxylic acids, carbonates, esters and the like), especially of C1-C20 and preferably of C1-C8, or mixtures thereof.

Examples of oxygenated compounds that may be used, without being limited thereto, are methanol, ethanol, n-propanol, isopropanol, butanol and isomers thereof, C4-C20 alcohols, methyl ethyl ether, dimethyl ether, diethyl ether, diisopropyl ether, formaldehyde, dimethyl carbonate, dimethyl ketone and acetic acid, preferably methanol, dimethyl ether (DME) and mixtures thereof, in particular with heavier alcohols.

The oxygenated compound(s) are obtained, for example, by conversion of biomass. This makes it possible to incorporate compounds of biological origin into the oligomerization products. The synthesis of oxygenated molecules from biomass may be performed via synthesis gas, a pyrolysis in the absence of oxygen, hydropyrolysis, a transetherification or an anaerobic or aerobic fermentation. The oxygenated molecules may be isolated or used as a mixture. The oxygenated molecules used may undergo a pretreatment in order to reduce their content of metal ions and of nitrogenous compounds.

Similarly, examples of sulfureous compounds are methanethiol, methyl sulfide, ethyl mercaptan (or thioethyl alcohol), ethyl sulfide, n-alkyl sulfides containing a C1-C10 n-alkyl group, and mixtures thereof.

Examples of halogenated compounds are ethyl monochloride, methyl monochloride, methyl dichloride, n-alkyl halides containing a C1-C10 n-alkyl group, and mixtures thereof.

The charge may be diluted using one or more inert diluents such as argon, helium, nitrogen, carbon monoxide, carbon dioxide, hydrogen, water, paraffins, alkanes (in particular methane, ethane and propane) or aromatic compounds, preferably water and nitrogen. The water may be injected in liquid or gaseous form.

The diluent(s) may represent from 1% to 95 mol % relative to the number of moles of the charge and of the diluents.

XTO Step of the Conversion Zone

The oligomerization treatment of a charge consisting solely of ethylene leads to highly branched hydrocarbons distilling from 165 to 350° C. The process according to the invention makes it possible to solve this problem by converting the light olefins, in particular of C2-C3, into heavier olefins (C4+) in the conversion zone, as described below.

In one embodiment, the effluents leaving the conversion zone are conveyed into at least one separation zone in which at least the C2-C3 olefins are separated out.

Advantageously, the C2-C3 olefins separated out are at least partially recycled with the charge for the conversion zone in order to increase the yield of C4+. These C2-C3 olefins, namely ethylene or ethylene and propylene, may thus be converted into heavier olefins (C4+). Given that only heavy olefins, especially C4+, make it possible to reduce the branching, this makes it possible to increase the yield of distillate in one run.

Moreover, the C3 olefins separated out may be at least partially recycled with the charge for the oligomerization zone.

In particular, propylene may be recycled into the oligomerization zone and the conversion zone, whereas ethylene will preferably be recycled only into the conversion zone.

As a variant, the C2-C3 olefins separated out may be recovered as final product for other applications, such as polymerizations, alkylations, etc.

The reaction conditions for the conversion step will be chosen so as to disfavor hydrogen transfer reactions leading to the formation of paraffins, aromatics and coke precursors.

In this step, the diluted or undiluted charge is placed in contact with a suitable catalyst, under conditions chosen so as to convert the heteroatomic organic compounds of the charge into predominantly light olefins, i.e. rich in C2-C4 olefins but comprising C2-C9 olefins.

The charge will preferably be in the vapor phase, but may also be in liquid form or in the form of a liquid-vapor mixture.

Advantageously, the temperature of the conversion zone is from 200 to 700° C.

At lower temperatures, the formation of olefins may be slower. At higher temperatures, the olefin yield may not be optimal. A temperature range of 300 to 600° C. will preferably be chosen.

The applied pressure may also be chosen over a wide range. Advantageously, the pressure of the conversion zone is from 5 kPa to 5 MPa and preferably from 50 kPa to 0.5 MPa. These pressures correspond to the partial pressure of the charge.

This conversion step may be performed, for example, in numerous types of entrained-bed reactor, or alternatively in fixed-bed or moving-bed reactors, preferably in fluidized-bed reactors.

The reaction will preferably be performed at a high weight hourly space velocity (WHSV) of the charge, for example from 0.1 h⁻¹ to 1000 h⁻¹.

The conversion zone may comprise one or more reaction zones, arranged in series or in parallel.

The catalyst may be regenerated after a certain time of use. This regeneration may be performed in the reactor itself or in a separate reactor by injecting a stream containing oxygen at a sufficiently high temperature to burn the coke deposited on the catalyst.

In the case of moving-bed or fluidized-bed reactors, part of the catalyst may be continuously or intermittently removed from the conversion reactor and conveyed to another reactor in order to be regenerated. After its regeneration, the withdrawn catalyst is continuously or intermittently returned to the conversion reactor.

In the case of fixed-bed reactors, the reactor is isolated from the installation, and the regeneration of the catalyst takes place in the reactor. In general, another reactor is then provided, which takes over the duty of the conversion to olefins. The reactor containing the regenerated catalyst is then placed on standby until regeneration of the catalyst in the second reactor is necessary.

The catalyst used will advantageously be a molecular sieve of reduced selectivity to form aromatic compounds, and with good selectivity to form C4+ olefins.

The term “molecular sieve” is defined as being a solid, porous material which has the property of acting as a sieve at the molecular scale. It is a class of catalyst that has the capacity of retaining certain molecules within its pores. Ideally, it has small, uniformly distributed pores. As a result, it has a high specific surface area. Zeolites are an example of molecular sieves.

In general, the molecular sieves that may be used may comprise acidic catalysts either of amorphous or crystalline aluminosilicate type, or of silicoaluminophosphate type. The molecular sieves that may be used are the following:

Silicoaluminophosphate molecular sieves: SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, metallic forms thereof, and mixtures thereof. The preferred sieves are SAPO-18, SAPO-34, SAPO-35, SAPO-44 and SAPO-47, in particular SAPO-18 and SAPO-34, and also the metallic forms thereof, and mixtures thereof.

Aluminosilicate molecular sieves: MFI (ZSM-5, silicalite-1, boralite C, TS-1), MEL (ZSM-11, silicalite-2, boralite D, TS-2, SSZ-46), ASA (amorphous silica alumina), MSA (mesoporous silica alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), ZSM-48, MFS (ZSM-57), MTW, MAZ, FAU, LTL, BETA MOR, and a family of microporous materials consisting of silicon, aluminum, oxygen and optionally boron.

The zeolite may undergo various treatments before its use, for example one or more of the following treatments: ion exchange, modification with metals, phosphorus, steam treatment, acid treatment or another dealumination process, surface passivation by deposition of silica.

The content of alkali metals, alkaline-earth metals or rare-earth metals is 0.05-10 by weight and preferably from 0.2 to 5 by weight. The metals will preferably be chosen from: Mg, Ca, Ba, Sr, La, Ce or mixtures thereof.

Oligomerization Step of the Oligomerization Zone

The oligomerization zone is fed with olefins at least partly constituting all or part of the effluent originating from the conversion zone, the oligomerization step being performed in the presence of oxygenated compounds.

Advantageously, in the effluent leaving the conversion zone, more than 80% by weight and preferably more than 85% by weight of the C4+ olefins are C4-C8 olefins. Among the C4+ olefins, butenes represent from 50% to 80% by weight.

The hydrocarbon-based charge used as charge for the oligomerization zone may also contain, in addition to part of the effluent from the conversion zone, a mixture of hydrocarbon-based effluents containing C2-C10 olefins derived from refinery or petrochemistry processes (FCC, vapor cracking, etc.). It may be a mixture of fractions comprising C3 FCC, C4 FCC, LCCS, LLCCS, Pygas, LCN, and mixtures, such that the content of linear olefins in the C5-fraction (C2-C5 hydrocarbons) relative to the total C2-C10 charge is not more than 40% by weight.

The total olefin content in the C5-(C2-C5) fraction relative to the total C2-C10 charge supplied for the oligomerization may be greater than 40% by weight if the isoolefins are present in an amount of at least 0.5% by weight.

The total content of linear olefins may be greater than 40% by weight relative to the total charge of C2-C10 if the linear C6+ olefins (C6, C7, C8, C9, C10) are present in an amount of at least 0.5% by weight.

This charge may especially contain olefins, paraffins and aromatic compounds in all proportions, in conformity with the rules described above.

The content of oxygenated compounds will be less than 70% by weight, preferably from 0.5% to 50% by weight and preferably from 1% to 30% by weight relative to the total charge treated in the oligomerization zone.

The oxygenated organic compounds contain at least one oxygen atom, such as alcohols, ethers, carbonyl compounds (aldehydes, ketones, carboxylic acids, carbonates, esters and the like), especially of C1-C20 and preferably of C1-C8, or mixtures thereof.

Examples of oxygenated compounds that may be used, without being limited thereto, are methanol, ethanol, n-propanol, isopropanol, butanol and isomers thereof, C4-C20 alcohols, methyl ethyl ether, dimethyl ether, diethyl ether, diisopropyl ether, formaldehyde, dimethyl carbonate, dimethyl ketone and acetic acid, preferably methanol or dimethyl ether (DME) and mixtures thereof, in particular with heavier alcohols.

Advantageously, the oxygenated compounds used during the oligomerization step are the same as those of the charge for the conversion step, especially methanol, dimethyl ether (DME) and mixtures thereof, in particular with heavier alcohols.

In general, the effluent from the oligomerization zone is conveyed into a separation zone, in order to separate, for example, the fractions into an aqueous fraction, C5-C9 (gasoline), C10-C12 (jet fuel) and C12+ (diesel). The fractions C5-C9, C10-C12 and C12+ may undergo drying.

Thus, the invention makes it possible especially to obtain a jet fuel (C10-C12) from oxygenated organic compounds, especially alcohols, of plant origin.

The fractions C10-C12 and C12+ separated from the effluent of the oligomerization process may undergo a hydrogenation in order to saturate the olefinic compounds and to hydrogenate the aromatic compounds. The product obtained has a high cetane number, and excellent properties for use as a fuel of jet or diesel type, or the like.

Advantageously, the effluents leaving the oligomerization zone are conveyed into at least one separation zone in which at least the C2-C4 olefins are separated out.

These C2-C4 olefins separated out may especially be at least partially recycled with the charge for the conversion zone so as to increase the yield of C4+ olefins, or sent into another separation zone.

It may also be possible to separate out the C5-C9 olefins, and then to recycle them with the charge for the conversion zone so as to increase the yield of olefins.

It may be envisioned to perform only the separation, and optionally the recycling, of the C5-C9 olefins, or alternatively in combination with the separation, and optionally the recycling, of the C2-C4 olefins.

Advantageously, before its treatment in the oligomerization zone, the charge undergoes selective hydrogenation and/or selective absorption.

The charge originating from the conversion zone may especially be, after hydrogenation, treated directly in the oligomerization zone, without prior fractionation of the heavy aromatic fractions.

Advantageously, the charge treated in the oligomerization step is placed in contact with a catalyst in the presence of a reducing compound, for example H₂. Preferably, the catalyst will then be an acidic or a difunctional metallic zeolite.

The oligomerization reaction will preferably be performed at an hourly space velocity (WHSV) of from 0.1 to 20 h⁻¹, preferably from 0.5 to 10 h⁻¹ and preferably from 1 to 8 h⁻¹. These velocities make it possible to obtain good conversion while at the same time limiting the adverse side reactions.

A multi-reactor system may be used, comprising cooling between the reactors for the purpose of controlling the reaction exothermicity, so that the temperature does not exceed a nominal temperature. Advantageously, the maximum acceptable temperature difference in each reactor will not exceed 100° C.

The reactor may be an isothermal or adiabatic fixed-bed reactor, or a series of reactors of this type, or alternatively one or more moving-bed reactors.

A typical moving-bed reactor is of the type with continuous catalytic reforming.

The oligomerization reaction may be performed continuously in a configuration comprising a series of fixed-bed reactors mounted in parallel, in which, when one or more reactors are operating, the other reactors undergo regeneration of the catalyst.

The temperature at the reactor inlet will advantageously be sufficient to allow a relatively high conversion, without being very high, so as to avoid adverse side reactions. The temperature at the reactor inlet will be, for example, from 150° C. to 400° C., preferably 200-350° C. and more preferably from 220 to 350° C.

The pressure across the oligomerization reactor(s) will advantageously be sufficient to allow a relatively high conversion, without being too low, so as to avoid adverse side reactions. The pressure across the reactor will be, for example, from 8 to 500 bara, preferably 10-150 bara and more preferably from 14 to 49 bara (bar, absolute pressure).

As regards the nature of the catalyst, a first family of catalysts comprises an acidic catalyst either of amorphous or crystalline aluminosilicate type, or a silicoaluminophosphate, in H+ form, chosen from the following list and optionally containing alkali metals or alkaline-earth metals:

MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), ZSM-48, MFS (ZSM-57), MTW, MAZ, SAPO-11, SAPO-5, FAU, LTL, BETA MOR, SAPO-40, SAPO-37, SAPO-41 and the family of microporous materials composed of silica, aluminum, oxygen and possibly boron.

Zeolite may be subjected to various treatments before use, which may be: ion exchange, modification with metals, steam treatment (steaming), acid treatments or any other dealumination method, surface passivation by deposition of silica, or any combination of the abovementioned treatments.

The content of alkali metals or rare-earth metals is from 0.05% to 10% by weight and preferentially from 0.2% to 5% by weight. Preferentially, the metals used are Mg, Ca, Ba, Sr, La and Ce, which are used alone or as a mixture.

A second family of catalysts that can be used comprises phosphate-modified zeolites optionally containing an alkali metal or a rare-earth metal. In this case, the zeolite may be chosen from the following list:

MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), MFS (ZSM-57), ZSM-48, MTW, MAZ, FA U, LTL, BETA MOR.

The zeolite may be subjected to various treatments before use, which may be: ion exchange, modification with metals, steam treatment (steaming), acid treatments or any other dealumination method, surface passivation by deposition of silica, or any combination of the abovementioned treatments.

The content of alkali metals or of rare-earth metals is from 0.05% to 10% by weight and preferentially from 0.2% to 5% by weight. Preferentially, the metals used are Mg, Ca, Ba, Sr, La and Ce, which are used alone or as a mixture.

A third family of catalysts that can be used comprises difunctional catalysts, comprising:
a support, from the following list: MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), MFS (ZSM-57), ZSM-48, MTW, MAZ, BETA, FAU, LTL, MOR, and microporous materials of the family ZSM-48 consisting of silicon, aluminum, oxygen and optionally boron. MFI or MEL (Si/Al>25), MCM-41, MCM-48, SBA-15, SBA-16, SiO₂, Al2O3, hydrotalcite, or a mixture thereof;
a metallic phase (Me) to a proportion of 0.1% by weight, the metal being selected from the following elements: Zn, Mn, Co, Ni, Ga, Fe, Ti, Zr, Ge, Sn and Cr used alone or as a mixture. These metal atoms may be inserted into the tetrahedral structure of the support via the tetrahedral unit [MeO₂]. The incorporation of this metal may be performed either by adding this metal during the synthesis of the support, or it may be incorporated after synthesis by ion exchange or impregnation, the metals then being incorporated in the form of cations, and not integrated into the structure of the support.

The zeolite may be subjected to various treatments before use, which may be: ion exchange, modification with metals, steam treatment (steaming), acid treatments or any other dealumination method, surface passivation by deposition of silica, or any combination of the abovementioned treatments.

The content of alkali metals, of alkali-earth metals or of rare-earth metals is from 0.05% to 10% by weight and preferentially from 0.2% to 5% by weight. Preferentially, the metals used are Mg, Ca, Ba, Sr, La and Ce, used alone or as a mixture.

Finally, the catalyst may be a mixture of the three families of catalysts described previously. In addition, the active phases may themselves also be combined with other constituents (binder, matrix) giving the final catalyst increased mechanical strength, or improved activity.

Although it is preferred to use different catalysts for the conversion and oligomerization zones, the same catalyst may be used in the two types of zone.

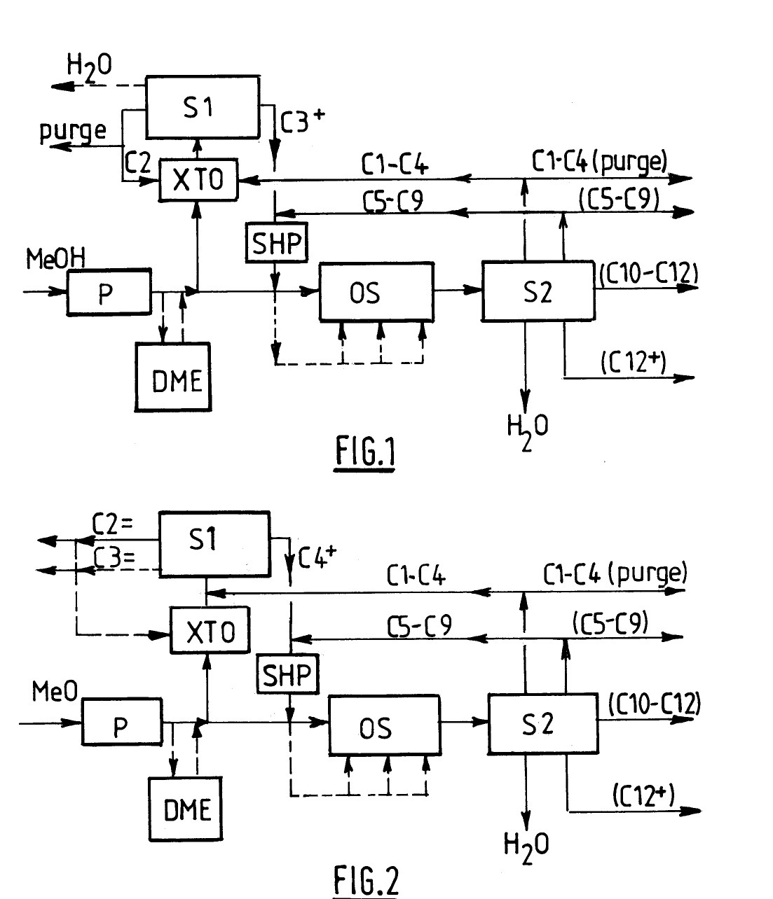

The invention is now described with reference to the examples and to the attached drawings, which are not limiting, in which FIGS. 1 to 4 schematically represent various embodiments of the process according to the invention.

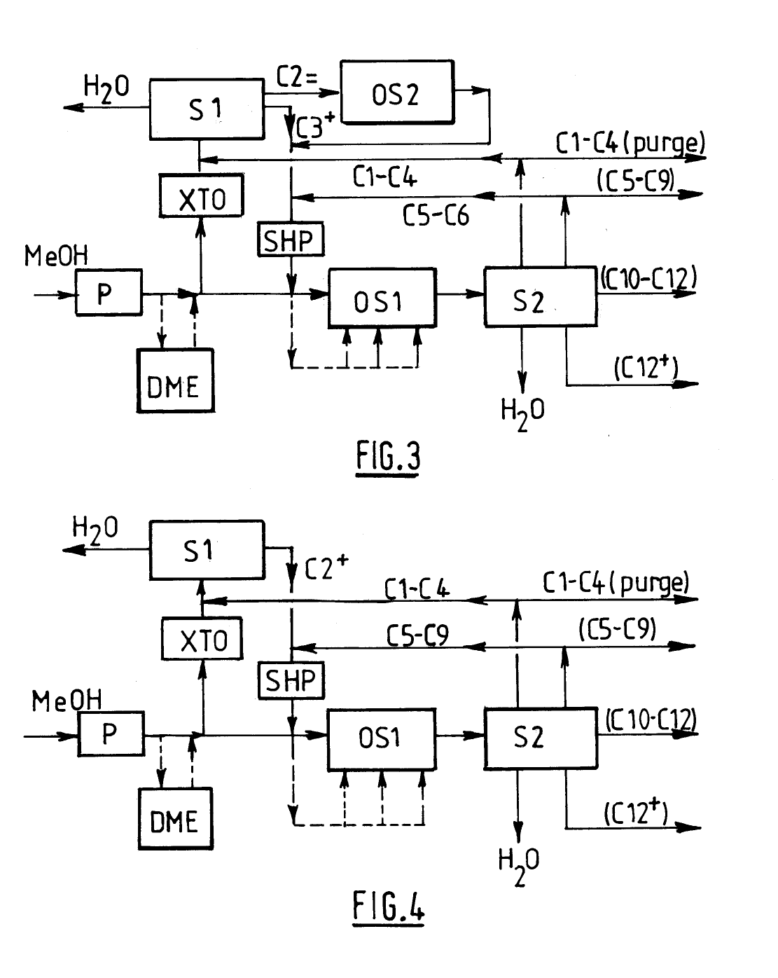

In each of the FIGS. 1 to 4:
XTO represents the zone for conversion of the heteroatomic organic compounds into olefins.
OS represents an oligomerization zone, FIG. 3 comprising two oligomerization zones, OS1 et OS2,
S1 and S2 represent separation zones,
SHP represents a zone of selective hydrogenation and/or of selective adsorption,
DME is a reactor for the production of dimethyl ether from methanol,
P represents a zone for purification of methanol MeOH.

On these figures, the dashed lines represent process options.

Needless to say, these embodiments may be performed with an oxygenated compound other than methanol, or a mixture of oxygenated compounds.

Each oligomerization zone represents, for example, an oligomerization reactor. The various embodiments described below may be combined together, especially the recycling modes thereof.

The scheme represented in FIG. 1 corresponds to a process in which the charge consisting of methanol MeOH, optionally mixed with DME, is treated in the XTO conversion zone. The effluent leaving this XTO zone is conveyed into the separation zone S1. In this zone S1, the C2 olefins are separated out and recycled into the XTO zone, the C3+ olefins are separated out, and the water is optionally removed.

The C3+ olefins, after selective hydrogenation (SHP), are then charged into the oligomerization zone OS as a mixture with the MeOH optionally mixed with DME. All of the MeOH and the DME may be added at the inlet of the zone OS and/or inside this zone (dashed lines).

The effluent leaving the zone OS is separated in the separation zone S2. In this zone S2, water is separated out, as are the C5-C9 gasolines, the C10-C12 jet fuel and the C12+ diesel. In addition, the light C2-C4 olefins are separated out and at least partly recycled as charge for the XTO conversion zone. Part of the C5-C9 olefins may also be recycled as charge for the selective hydrogenation zone SHP.

The process represented schematically in FIG. 2 differs from that of FIG. 1 by the recycling performed.

The light C2-C4 olefins derived from the separation zone S2 are recycled, not as charge for the zone XTO, but downstream of this zone, as charge for the separation zone S1.

Moreover, the separation zone S1 separates the olefins into C2= and C3=(ethylene and propylene), which can either form finished products, or be recycled as charge for the XTO zone (or even both), whereas the C4+ olefins are conveyed into the oligomerization zone via the selective hydrogenation zone (SHP).

The process represented schematically in FIG. 3 differs from that of FIG. 2 by the presence of two oligomerization zones OS1 and OS2 .

The zone OS1 corresponds to the zone OS of FIG. 2.

In this process, the separation zone S1 separates the olefins into C3+ and C2=(ethylene). The C2 olefins are treated in the second oligomerization zone OS2, the effluent of which is mixed with the C3+ olefins, upstream of the zone SHP, in order to constitute the charge for the oligomerization zone OS1.

The process represented schematically in FIG. 4 differs from that of FIG. 2 by the recycling performed.

In this process, the separation zone S1 separates the water from the C2+ olefins, which are then treated in the oligomerization zone OS1 after passage through the zone SHP.

In all cases, the separation zones S1 and S2 may be one and the same zone.

### EXAMPLES

### Example 1

### Preparation of Catalyst A

A sample of phosphated zeolites ZMS-5 prepared in accordance with an example adapted from document EP 2025402 A1 starting with HZMS-5 (Si/Al=13) synthesized without “templates” was extruded with a silica sol with a low sodium content and a phosphated xonotlite (Ca/P˜1) and 2-3% of extrusion additives. The catalyst obtained contains up to about 40% by weight of zeolites. The dried extruded catalyst was washed with an aqueous solution at room temperature, and then dried at 110° C. for 16 hours and calcined at 700° C. for 2 hours.

The product thus obtained is named catalyst A.

### Examples 2 to 4

### Reaction Tests

In these tests, a charge of methanol mixed with ethylene was converted into olefins. The addition of ethylene to the methanol charge (Examples 3 and 4) corresponds to the recycling of ethylene as charge for the XTO zone, as represented schematically in FIG. 1 described previously.

The charge of methanol and ethylene is introduced into a descending-stream fixed-bed reactor comprising catalyst A in the form of grains (35-45 mesh).

Before the tests, the catalyst was heated under a stream of nitrogen (5 Nl/h) up to the reaction temperature.

Analysis of the product obtained was performed online by gas chromatography, the chromatograph being equipped with a capillary column.

A substantially complete conversion of the methanol was observed, the catalyst showing stable performance.

Table 1 collates the reaction conditions, the charges tested and the product obtained after running the reactor for 3 hours.

The experimental conditions were determined so as to optimize the yield of total olefins, especially without recycling of ethylene (Example 2).

Examples 3 and 4 differ by the composition of the charge.

These examples show that ethylene can be fully converted into heavier olefins (C3+) in an XTO zone without any detectable loss in the total content of olefins. On the other hand, the ethylene reacts relatively sparingly in the oligomerization zone.

TABLE 1

| | Example | | |
|---|---|---|---|
| | 2 | 3 | 4 |
| | Composition of the charge (on C basis, weight %) | | |
| Ethylene | 0 | 28.6 | 16.8 |
| Methanol | 100 | 71.4 | 83.2 |

Experimental conditions

| | |
|---|---|
| WHSV, h⁻¹, C-basis | 1.2 |
| T, ° C. | 550 |
| P, bara | 1.5 |

Composition effluent (on carbon basis), weight %

| | | | | effluent derived |
|---|---|---|---|---|
| | | | total effluent | from methanol |
| Ethylene | 10.2 | 21.4 | 16.8 | 0 |
| Propylene | 40.7 | 35.1 | 38.0 | 45.6 |
| C4+ Olefins | 36.3 | 33.4 | 25.1 | 42.2 |
| Total Olefins | 87.2 | 29.8 | 89.9 | 87.9 |

### Example 5

### Comparative

In the same way as in the preceding example, the reactor used is a descending-stream fixed-bed reactor comprising catalyst A in the form of grains (35-45 mesh).

Before the tests, the catalyst was heated under a stream of nitrogen (5 Nl/h) up to the reaction temperature.

Analysis of the product obtained was performed online by gas chromatography, the chromatograph being equipped with a capillary column.

The pure methanol was charged into the reactor at 550° C., under a pressure P=1.5 bara. These conditions make it possible to optimize the yield of C3+ olefins in a single run.

A substantially total conversion of the methanol was observed, the catalyst showing stable performance.

Table 2 collates the reaction conditions, the charges tested and the product obtained after running for 5 hours.

These examples show that the recycling of ethylene into the XTO zone (Example 4) is beneficial for obtaining C3+ relative to maximization of the yield of these products in a single run (one-through, Example 5).

TABLE 2

| | Example | | |
|---|---|---|---|
| | 5 (comp) | 2 | 4 |
| Description | MTO one- | MTO | MTO + |
| | through | | Ethylene |
| Mode | C3+ olefin | Total | |
| | Max | olefins max | |

Composition of the charge (carbon basis, weight %)

| | | | |
|---|---|---|---|
| Ethylene | 0 | 0 | 16.8 |
| Methanol | 100 | 100 | 83.2 |

Experimental conditions

| | | | |
|---|---|---|---|
| WHSV, h⁻¹, C-basis | 2.5 | 1.2 | 1.2 |
| T, ° C. | 500 | 550 | 550 |
| P, bara | 1.5 | 1.5 | 1.5 |

Composition of the effluent derived from methanol (carbon basis),
weight %

| | | | |
|---|---|---|---|
| Ethylene | 4.8 | 10.2 | 0 |
| Total C3+ olefins | 82.8 | 77.0 | 87.9 |
| Total olefins | 84.9 | 87.2 | 87.9 |

### Example 6

### Preparation of Catalyst B

A sample of silicalite (MFI, Si/Al=200) in NH₄ form was calcined at 550° C. for 6 hours in order to convert it into H+ form.

The product thus obtained is named catalyst B.

### Examples 7-14

### Oligomerization Tests

Reaction tests were performed in a descending-stream fixed-bed tubular reactor into which was precharged catalyst B in the form of grains (35-45 mesh).

Before the tests, catalyst B was activated at 550° C. for 6 hours under a stream of nitrogen.

The charges for the oligomerization tests were prepared by mixing n-pentane or 1-hexene with methanol.

The charges containing oxygenated and hydrocarbon-based compounds were placed in contact with catalyst B at an inlet temperature of 300° C., under P=15 barg (P (barg)=P bar−Patm (˜1 bar)), and with an hourly space velocity (WHSV) for the charge of 4 h⁻¹.

Analysis of the products obtained was performed online by gas chromatography, the chromatograph being equipped with a capillary column.

At the reactor outlet, the gaseous phase, the liquid organic phase and the aqueous phase were separated. No recycling was performed.

Table 3 collates the results of the tests. The methanol was taken into account in the olefins (—CH₂—).

These results show the beneficial effect of the presence of methanol on the yield of heavy fraction and the decrease in the yields of light fractions (C1-C5).

TABLE 3

| | Examples | | | |
|---|---|---|---|---|
| | 7 (comp) | 8 | 9 | 10 |
| Catalyst | B | | | |
| Charge | 70% 1- | 10% | 30% | 50% |
| | hexene | methanol | methanol | methanol |
| | 30% n- | 60% 1- | 40% 1- | 20% 1- |
| | pentane | hexene | hexene | hexene |
| | | 30% n- | 30% n- | 30% n- |
| | | pentane | pentane | pentane |

| | |
|---|---|
| TOS, h | 5 |
| WHSV, h⁻¹ | 4 |
| P, barg | 15 |
| T, ° C. | 300 |

Yields of olefins, weight %

| | | | | |
|---|---|---|---|---|
| C1-C5 | 17.8 | 12.1 | 11.9 | 13.9 |
| (+DME) | | | | |
| C6-C11 (of | 42.6 | 44.6 | 34.2 | 40.5 |
| which 1- | | | | |
| hexene) | | | | |
| 1-hexene | 23.3 | 20 | 30.3 | 19.8 |
| C12+ | 16.3 | 23.3 | 23.6 | 25.8 |

TOS: test time

1. Process for producing distillate, hydrocarbons containing 10 or more carbon atoms, from a charge of heteroatomic organic compounds comprising at least one heteroatom chosen from oxygen, sulfur and halogen, alone or in combination, in which the treatment of the charge comprises at least one step of conversion of the heteroatomic organic compounds into olefins performed in a first conversion zone, and, in at least a second oligomerization zone, a step of oligomerization of olefins originating at least partly from the conversion zone, in the presence of at least 0.5% by weight of oxygenated compounds, in order to produce a distillate.

2. Process according to claim 1, in which the charge for the oligomerization step comprises, besides olefins originating from the conversion zone, C3-C10 olefins.

3. Process according to claim 1, in which the heteroatomic organic compounds are organic compounds containing at least one oxygen atom, especially of C1-C20 and preferably of C1-C8.

4. Process according to claim 3, in which the oxygenated compound is chosen from methanol, ethanol, n-propanol, isopropanol, butanol and isomers thereof, C4-C20 alcohols, methyl ethyl ether, dimethyl ether, diethyl ether, diisopropyl ether, formaldehyde, dimethyl carbonate, dimethyl ketone and acetic acid, and mixtures thereof.

5. Process according to claim 3, in which the oxygenated compounds of the oligomerization zone are the same as the heteroatomic compounds of the conversion zone.

6. Process according to claim 1, in which the content of oxygenated compounds will be less than 70% by weight, preferably from 0.5% to 50% by weight and preferably from 10% to 30% by weight relative to the total charge treated in the oligomerization zone.

7. Process according to claim 1, in which the charge is diluted with at least one inert diluent, the diluent content of the total charge being from 1 to 95 mol %.

8. Process according to claim 1, in which the effluents leaving the conversion zone are conveyed into at least one separation zone in which at least the C2-C3 olefins are separated out.

9. Process according to claim 8, in which the C2-C3 olefins separated out are at least partially recycled with the charge for the conversion zone.

10. Process according to claim 8, in which the C3 olefins separated out are at least partially recycled with the charge for the oligomerization zone.

11. Process according to claim 1, in which the effluents leaving the oligomerization zone are conveyed into at least one separation zone in which at least the C2-C4 olefins are separated out.

12. Process according to claim 11, in which the C2-C4 olefins separated out are at least partially recycled with the charge for the conversion zone or sent into another separation zone.

13. Process according to claim 11, in which, in the separation zone, the C5-C9 olefins are separated out and are then recycled with the charge for the conversion zone.

14. Process according to claim 1, in which, before its treatment in the oligomerization zone, the charge undergoes a selective hydrogenation.

15. Process according to claim 1, in which the temperature of the conversion zone is from 200 to 700° C. and preferably from 300 to 600° C.

16. Process according to claim 1, in which the pressure of the conversion zone is from 5 kPa to 5 MPa and preferably from 50 kPa to 0.5 MPa.

17. Process according to claim 1, in which the reaction of the oligomerization zone is performed at an hourly space velocity (WHSV) of the charge of from 0.1 to 20 h⁻¹, preferably from 0.5 to 10 h⁻¹ and preferably from 1 to 8 h⁻¹.

18. Process according to claim 1, in which the temperature at the inlet of the reactor(s) of the oligomerization zone is from 150 to 400° C., preferably 200-350° C. and more preferably from 220 to 350° C.

19. Process according to claim 1, in which the pressure across the reactor(s) of the oligomerization zone is from 8 to 500 bara, preferably 10-150 bara and more preferably from 14 to 49 bara.
